(19) United States
(12) Patent Application Publication
(10) Pub. No.: US 2024/0021296 A1
(43) Pub. Date: Jan. 18, 2024

(54) Systems and methods for easy identification, modification and progress-Tracking of Variables relating to appropriateness, safety, effectiveness, risks and benefits of a medicinal treatment combination

(71) Applicant: Robert Moore, Hereford, AZ (US)

(72) Inventor: Robert Moore, Hereford, AZ (US)

(21) Appl. No.: 18/219,088

(22) Filed: Jul. 6, 2023

Related U.S. Application Data
(60) Provisional application No. 63/358,793, filed on Jul. 6, 2022.

(51) Int. Cl. G16H 40/20; G16H 15/00

(52) U.S. Cl. CPC G16H 40/20; G16H 15/00

(57) ABSTRACT

This invention presents a method for the arrangement of information corresponding to a combination of treatments for electronic use, being derived from a method for the organization and storage of medicines and supplements in a storage organizer, which can be readily reviewed by patients and healthcare providers to facilitate Medication Therapy Management Services.

### CROSS-REFERENCE TO RELATED APPLICATIONS

This application claims the benefit of PPA Ser. No. 63/358,793.

### BACKGROUND

The following is a tabulation of some prior art that presently appears relevant:

US Patents

| Patent Number | Kind Code | Issue Date | Patentee |
|---|---|---|---|
| 11,031,108 | B2 | 2021 Jun. 8 | Tsung-Hsiang Liu, et al |
| 5,924,074 | | 1999 Jul. 13 | Evans, Jae A. |
| 11,282,107 | B1 | 2022 Mar. 22 | Yi Liu, et al. |
| 5,868,669 | | 1999 Feb. 9 | Lliff, Edwin |

U.S. Patent Application Publications

| Publication Nr. | Kind Code | Publ. Date | Applicant |
|---|---|---|---|
| 20090144089 | A1 | 2009 Jun. 4 | Heywood, Benjamin et al. |
| 20020095584 | A1 | 2002 Jul. 18 | Royer, Barry Lynn, et al. |

The field of medicine is complicated with hundreds of medicinal treatments, including prescriptions medicines, over-the-counter supplements, and natural supplements. While it is already challenging to account for the profile or characteristics of each treatment, when used in combination, a new profile can emerge with characteristics that are significantly different. Furthermore, new medicinal treatment options and combinations are regularly entering the market. These factors indicate the need for best-practices in teaching about these profiles or characteristics to help ensure appropriate, safe and effective combinations given risk and benefits, helping to empower recipients with understanding that leads to improved adherence, and ultimately, positive health outcomes.

Medication Therapy Management (MTM) is a service that helps to ensure appropriateness, safety and effectiveness of medicinal treatments based on known risks and benefits, taking into account medicinal treatment characteristics and the best of what is known about treatments combinations. These characteristics include the name of the medicine or supplement, its strength, any prescription number, the date it was prescribed or first started, the prescriber or manufacturer, the shape, color and/or markings on the tablet, capsule or supplement, the indication of the treatment or why it was prescribed, the intended result the treatment produces or should produce, any contraindications, special considerations, monitoring parameters, and possible side effects. The present art facilitates review, optimization and reproduction of these characteristics, as well as implementation of a storage organizer and method that further facilitates medicinal treatment understanding and medicinal treatment characteristics.

The art of U.S. patent application Ser. No. 15/276,337 aims to reduce treatment complexity and aid recipient understanding through a storage organizer and the accompanying method of using it. This method results in the organization of treatments into columns of compartments on a weekly medicine organizer, wherein each medicinal treatment is afforded its own column of compartments spanning each day of the week arranged as seven rows. A means for displaying indicia is situated beneath the compartments, slidably disposing rows of information corresponding with each column on the organizer. By slidably disposing the display means, a user can readily identify the profile or characteristics of their medicinal treatments according to each corresponding field of information. No other organizer has taught or implemented this practice or produced such results.

The present art is another new and unexpected result of the aforementioned art. Because the method of the aforementioned art results in a specific organization of treatments, that arrangement can be entered into electronic form for further editing, storage and reproduction. This allows systemization towards establishing best treatments, enabling providers to more easily collaborate on the value of a given combination, recording the results in a manner easily accessible to other providers in a chronologically. Furthermore, patients or recipients using the aforementioned art can readily identify work that has already been completed on a given combination by a variety of healthcare providers, especially with respect to over-the-counter supplements and other natural supplements, which are not always reviewed by providers, but can alter the way other medicinal treatments work, altering the course of disease for better or for worse.

The present art consists primarily in a method of electronically storing the specific arrangement of treatment options in accordance with how they are stored in the aforementioned art. In being systematic, it is reducible to an algorithm that can results in only few keyboard characters to produce a URL, which can then load a webpage consisting of the aforementioned treatment characteristics as arranged into a template on the display means of the aforementioned art for electronic display, such as a tablet computer, or for printing on a sheet of paper, both of which serve as means for displaying indicia. However, in its most basic form, which is arguably the best form, no algorithm is used and the URL consists of the typed out generic names of all the medicinal treatments. This method is preferred because it is easy to understand. Similarly, any person who can spell and enter information from a keyboard into a computer, can access a template used as a display means for the art of Ser. No. 15/276,337, using that template as a printable piece of paper or for display on a tablet computer.

While other art forms address the use of computers for entry and analysis of treatment information, none specifically address a method that directly coincides with the physical storage of treatments according to their administration times. As an example, the art of Evans, U.S. Pat. No. 5,924,074, presents a “medical record system that creates and maintains all patient data electronically.” Accordingly, FIG. 24 presents a computer network while FIG. 19 presents a webpage with a series of tabs that include “progress notes,” “patient data,” “medication date,” “laboratory,” and “practice guidelines.” FIG. 11 presents a flow-chart showing a process for “patient data capture,” which includes “clinical data capture,” “progress notes,” “medication data capture,” “practice guidelines,” and “progress notes.” However, the art form does not suggest or teach a direct and corresponding relationship between a specific storage arrangement of medicinal treatments on a storage organizer according to their administration times and a method of electronically reproducing those arrangements for storage, retrieval and editing. This feature further enables comprehensive review and standardization of medicinal characteristics that correlate linearly with the medicinal treatments.

The art of Lliff, U.S. Pat. No. 5,868,669, entitled “Computerized Medical Diagnostic and Treatment Advice System,” also demonstrates a flow-chart with similar boxes such as “patient response,” “patient medication,” “evaluation process,” and “treatment table,” as shown in FIGS. 6, 7d, but also does not appear to address any specific process or steps by which these activities occur to produce a result. The situation is similar for the art of Rubin, entitled “System and User Interface Supporting Concurrent Application Initiation and Interoperability.” In FIG. 4 of this art, a flowchart is shown describing a process whereby a webpage is modified, but does not contain any specific information concerning how that webpage is classified, or based on the content, how it is edited, specifically with respect to favorable patient outcomes in view of medicine appropriateness, safety and effectiveness. Arguably, the specific method of classifying a webpage, and its content, are determining factors in the success of an electronic system of medication therapy management. In contrast, the art of Heywood, US. Pat. App. 2009/0144089 demonstrates the generation of a “graphical element” in FIG. 1, as opposed to a webpage, while FIG. 2 denotes a flowchart with boxes such as “analyze profiles,” and “analyze measurements,” again not describing an actual system whereby this occurs to optimize patient outcomes in view of medicinal treatments. However, the art of Heywood does present an electronic system whereby a user may review trends of input information about a specific medicine treatment, such as checking the box of FIG. 3, which states “I'm feeling great!,” or “I'm feeling bad,” in the context of using “Riluzole 5 mg” twice a day versus once a day. Still, such a system does take advantage of public familiarity with the use of a URL, nor does the system correspond to the physical arrangement of stored medicinal treatments, nor does it comprehensively addresses all of the individual profiles or medicinal treatment characteristics in one template. This difference also applies to the art of Tsung-Hsiang of U.S. Pat. No. 11,031,108 for “Medicine Management Method and Medicine Management Device.”

With respect to another new and unexpected result of the present art, insofar as a given combination produces a unique profile, for example a prescription medicine in combination with a natural supplement that reduces the blood levels of the prescription medicine, any fields of information that are affected, such as the drug monitoring field of the prescription medicine and the special considerations field of the natural supplement, can be edited in the corresponding column of information and stored electronically for future reproducibility and comprehensive review in the same template because the URL directly corresponds to a webpage hosting information about that combination, in accordance with the standard of the art for webpage construction and management. Furthermore, a one or two-digit number, or one or two letters, or a combination of letters and numbers, may follow the name of that treatment causing a problem, in accordance with a specific algorithm, to correspond to a degree of effect on the blood levels, or in the case of an adverse effect, the degree to which the effect impacted the patient, whether respect to feeling ill or having a rash, or the degree to which the adverse effect influenced the patient to stop the medication before restarting it, such as the number of days. Nonetheless, a near infinite number of algorithms can be used with respect to determining which two specific alpha, numeric, or alphanumeric characters to use, and, appending a URL with information specific to a webpage, another function, or parameter, in itself is not novel, as demonstrated by the art of Liu et al. of U.S. Pat. No. 11,282,107 for “Tracking Advertisements Using a Single URL Without Redirection.”

### SUMMARY

The method of the art form comprises the steps of assigning a URL for the creation of a webpage according to the physical arrangement of items stored on a storage organizer, more specifically the organizer of art U.S. patent application Ser. No. 15/276,337. This method, even without the use of the organizer of art form Ser. No. 15/276,337, which has been the basis for discovering this method, facilitates patient understanding of, and adherence to, medicinal treatments, including those administered according to a specific interval of time, such as on an “as needed” basis.

In one example of the art, the system encompasses the names of the substances administered as an .html file with a special character dividing the names; in another example the .html file includes names as well as administration times; in a third example, names, administration times and indication is included in the .html file. In yet another example of the system, abbreviations are used to truncate the time needed to enter popular combinations of treatments. In the first example, patients who can spell the name of their treatments as generic substances can enter the URL; in the second example they can begin to consider what administration times may be most beneficent, or from the resulting webpage of the first example, click on administration times that are most popular with that specific combination, which can then result in a function to append that section of the URL corresponding to that treatment or combination of treatments with specific times according to the standard of the art for webpage management; in the third example, they can consider the indication they have in mind for a specific treatment, or they can be directed from the second example to click on a corresponding indication that appends the URL with that information as previously described for the second example. From any of these first, second, or third examples, an abbreviated HTML page can be generated according to an algorithm that can allow easier access in the future, though such abbreviation is not necessary and not preferred in that the written description of medicinal treatments is more readily understandable and memorable, except insofar as a treatment combination exceeds the character limit for a URL entry, currently 2,048 characters. Nor is it necessary for substances names to be in English, the language this application has been written in, as each language likely has a standard generic name for every medicinal substance, including a Latin name for a substance derived directly from a living thing.

Since the system encompasses an electronic file readily accessible from an online server, it can then be modified at any time to include the different strengths of the medicines or supplements, durations of treatment time for the combination in order to establish long-term benefits and risks, the manufacturer or appearance of any individual component of the treatment to establish trends related to safety and efficacy, risks and benefits, such manufacturing best-practices and variations that can be found in various natural substances when obtained at different stages of a life-cycle, the intended indication of the treatment combination, measurable results the treatment combination produces, special considerations or monitoring parameters of the treatment combination, possible side effects, noted side effects, quantitatively measured side effects, genetic connotations, which could help to address specific populations at risk for individual or combinations of treatments based on metabolism or autoimmune disorders, and laboratory result connotations, which could help to identify patients more likely to benefit from the combination, or at higher risk for complications with a specific treatment combination. Note again that many of these characteristics have been previously addressed in the art of U.S. patent application Ser. No. 15/276,337, and are readily observable for any user of the organizer presented in that art form through the slidable, interchangeable, and readily accessible display means.

The process begins by obtaining the comprehensive combination of treatments of a patient or animal. This would include all written prescriptions, whether from the same of different prescribers, all over-the-counter supplements, and any substances obtained directly from nature, such as plant parts used as herbal supplements. Once the combination is obtained, the next step involves listing all of the names of those substances by using the generic name of the substance, as opposed to the brand or trade name in the case of a prescription or over-the-counter substance, and the Latin name of the natural source for any substance or supplement derived directly from nature. From this step a list can be generated. At this point the list can be used to place the corresponding items in a storage organizer according to an administration time that corresponds with an appropriate administration time. This then results in a list according to administration times from earliest time in the day to the latest, and includes frequencies of administration given on an as needed basis, such as every eight hours as needed. This list then forms the basis of a URL as a direct entry into a URL field of an internet browser according to whatever domain name is hosting the webpages that correspond to that arrangement, wherein the names may be joined together or preferably, spaced by an alphanumeric character, such as the dash character. However, some users of the preceding step may find it difficult to determine which administration times are most appropriate. In this case the list can be alphabetized and the alphabetized list can then be entered directly as a URL. However, it need not be alphabetized as there are a finite number of combinations that can be generated from such a list, and once an initial list has been created, the process of arranging the names in any order can be automated in accordance with the standard of the art, including an arrangement specific to a healthcare provider's intention for a specific purpose, such as medicines or substances they have specialized in, including an automatic omission of those not relevant to a present investigation. In either case, the result of the list can then be typed directly into an internet browser on any domain hosting webpages that a customer has chosen as being worthy of providing their medicine management services and that seeks to utilize the present method and the inventive concepts addressed in U.S. patent application Ser. No. 15/276,337.

Because the method is simple and easy to understand, anyone who can type the names of the substances they are taking can go to a webpage hosted on a server to have the server return information relevant to their specific treatment combination, being redirected if necessary, depending on the order they typed the names into the URL field, to a Combination Master Page for that combination. Nonetheless, because each generic name also has a trade or brand name associated with it, once a generic name has been used in the generation of a list, a process can be automated in accordance with the current skill in the art of computer programming and automatic webpage generation, to substitute every possible brand and trade name, including those of foreign countries and in foreign languages and using foreign alphabet characters or symbols, so that should a patient forget to change the name, or type in a foreign name using foreign characters that correspond to the same substance, they will still reach the same Combination Master Page.

The use of a Combination Master Page can further allow all foreign countries to benefit from a specific evaluation completed by healthcare providers, providing they allow a server to function in this capacity by storing at least one page corresponding to at least one list, and utilize the same or similar software for the automation of list name alternatives. At any rate, similarly, since the combination has a finite number of possibilities with respect to the ordering of the names, as well as indications and administration times, each possibility can allow the creation of a unique and corresponding webpage URL, which could then be used to redirect to the Combination Master Page whenever a user enters any ordering of the combination into the URL field of their browser, thus systemizing the combination and allowing direction to the Combination Master Page.

A hierarchy of permission can enable the best Combination Master Page to emerge, with a review board being likely the highest authoritative permission, depending on the size of the organization and resources devoted to the development of the process, as well as the general acceptance of the organizer of U.S. patent application Ser. No. 15/276,337, and the associated methods, as a preeminent example of excellence in the standard of care. In both cases, the process of understanding treatments, treatment combinations, treatment characteristics, and the process of Medication Therapy Management, is facilitated.

Furthermore, the ongoing final Combination Master Page becomes a medium through which a discussion of the aforementioned topics pertaining to the safety and efficacy, risks and benefits of the combination can occur, thus advancing the field of medicine as a whole. Furthermore, this page can be used to print out the arranged combination on a piece of paper, or uploaded to a means for displaying indicia, as addressed in the art of U.S. patent application Ser. No. 15/276,337, to facilitate use of the organizer, optimizing the presentation of information relevant to their safety and effectiveness, risks and benefits of the treatment combination, while also helping to improve adherence through an easy view of each day's treatments and administration times. Similarly, each column of substance's characteristic information can be positioned directly adjacent to the corresponding column of stored substance to assimilate understanding and retention.

### DRAWINGS-FIGURES

FIG. 1 is a bird's eye view of an embodiment of the organizer of U.S. Pat. App adjacent to a means for displaying indicia.

FIG. 2 is a bird's eye view of an embodiment of the organizer of U.S. Pat. App adjacent to a means for displaying indicia with indicia denoting a combination of medicines and supplements as a treatment plan.

FIG. 3 is a bird's eye view of the organizer of U.S. patent application Ser. No. 15/276,337 with the means for displaying indicia inserted into the organizer.

FIG. 4 is a in bird's eye of the means for displaying indicia presented in FIGS. 2 & 3, displaying a treatment plan with the organizer of U.S. patent application Ser. No. 15/276,337.

FIG. 5 presents an example of a treatment combination as a file name that corresponds with the treatment plan displayed in FIG. 4, with medicinal treatments arranged according to administration time.

FIG. 6 presents another example of a treatment combination as a file name that corresponds with the treatment plan displayed in FIG. 4, with medicinal treatments arranged alphabetically.

FIG. 7 presents another example of a treatment combination as a file name that corresponds with FIG. 2, which is the same treatment plan displayed in FIG. 4, wherein administration times are appended to the URL in AM/PM time format.

FIG. 8 presents another example of a treatment combination as a file name that corresponds with FIG. 2, which is the same treatment plan displayed in FIG. 4, wherein administration times are appended to the URL in military time format.

FIG. 9 presents another example of a treatment combination as a file name that corresponds with FIG. 2, which is the same treatment plan displayed in FIG. 4, wherein indications as well as administration times, including “as needed” use signified by the letters “PRN,” are appended to the URL in military time format.

FIG. 10 presents another example of a treatment combination as a file name that corresponds with FIG. 3, wherein indications as well as administration times are appended to the URL in military time format, as well as “as needed” use of a treatment according to an interval, for example “PRNQ8H” for “every eight hours as needed,” as well as “WILU,” signifying “with lunch.”

FIG. 11 presents a flowchart demonstrating the steps involved with the system of the present art.

### DRAWINGS

### Reference Numerals

100 an embodiment for an organizer
102 an administration time of “7 am” as displayed on an organizer
104 an administration time of “7:30”
106 an administration time of “With Lunch”
108 an administration time of “7 pm” as needed”
110 an administration time of “two times a day
112 an administration time of “every eight hours as needed”
120 another embodiment for an organizer
200 means for displaying indicia displaying empty rows corresponding to columns on the organizer, wherein said rows are divided into columns for distinct categories of characterizing information
202 displayed indicia on means for displaying indicia as though pulled out partially from organizer

### DETAILED DESCRIPTION

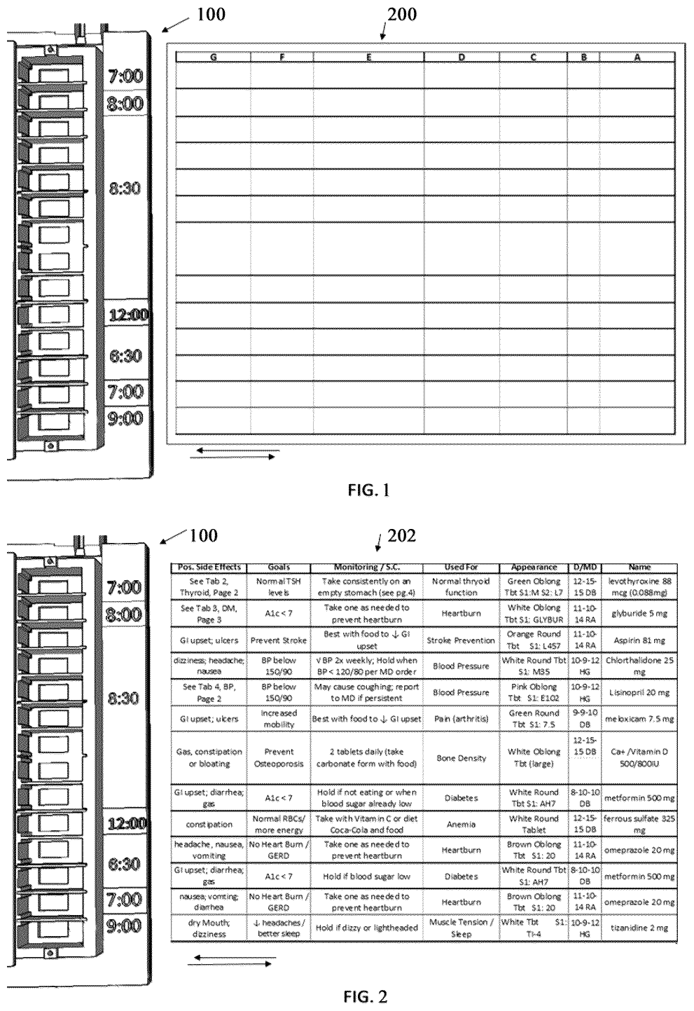

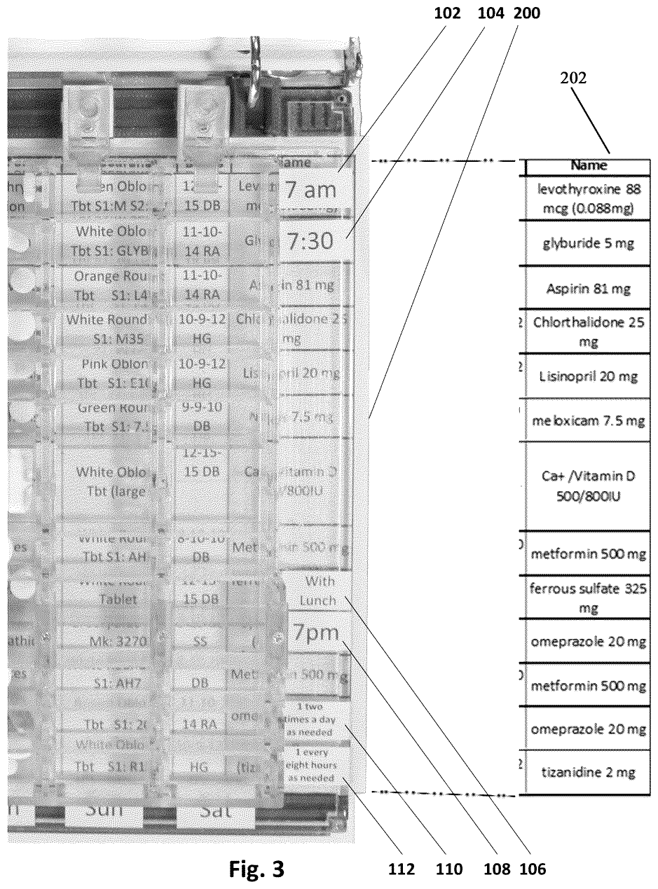

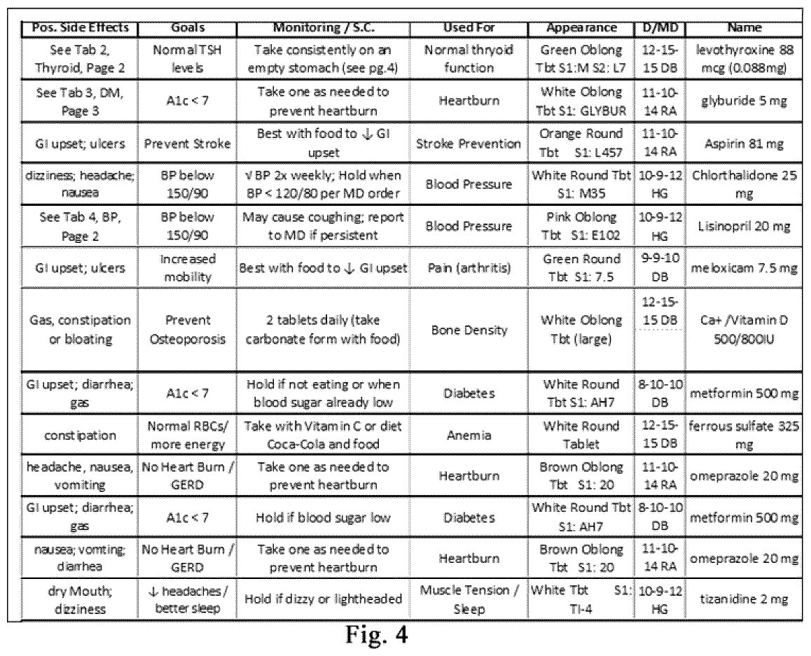

FIG. 1 present a bird's eye view of an organizer 100 adjacent to a means for displaying indicia 200. FIG. 2 is a bird's eye view of an organizer 100 adjacent to a means for displaying indicia 202 with indicia denoting a combination of medicines and supplements as a treatment plan. FIG. 3 is a bird's eye view of an organizer 120 with the same means for displaying indicia 202 inserted into the organizer but also shown as though partially slid out of the organizer. Note on the organizer that there are vertically descending indicium depicting administration times of: “7 am” 102, “7:30” 104, “With Lunch” 106, “7 pm” 108, “two as needed” 110, “every eight hours as needed” 112, respectively, which correspond with columns of compartments on the organizer. These administration times are included in the system of creating file names and presented in FIGS. 8-10. FIG. 4 is a in bird's eye of the same means for displaying indicia 202 with the same indicia but without an organizer to allow a closer view of said indicia.

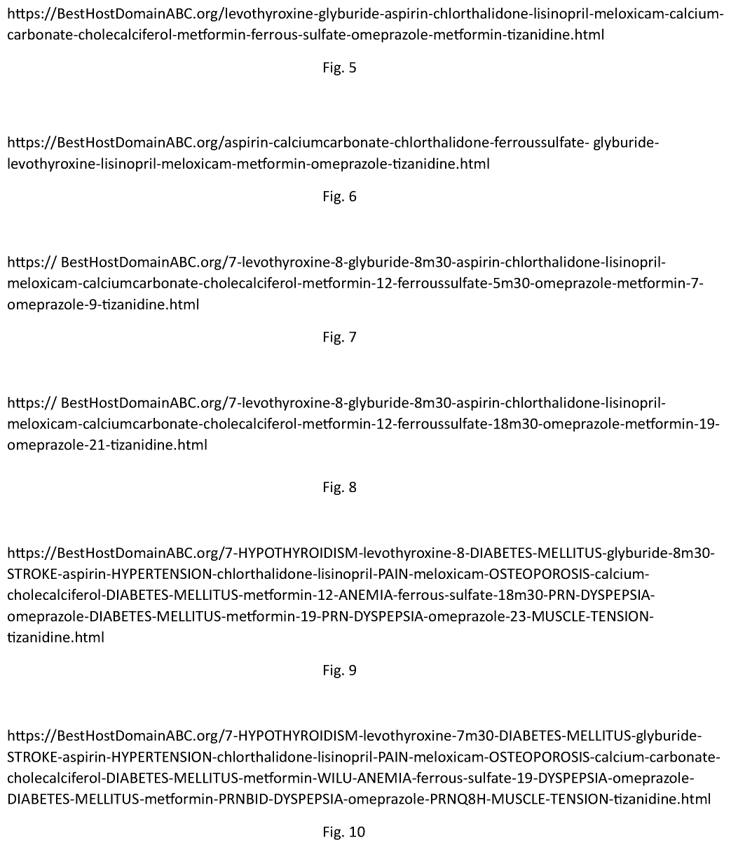

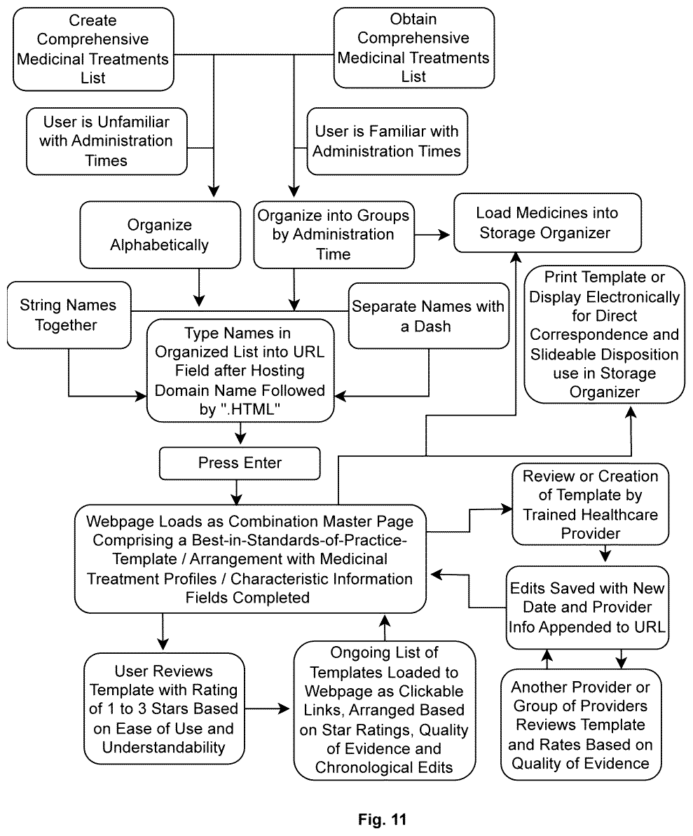

FIGS. 5-10 depict file names created in accordance with the methods presented earlier, and the system of organizing medicines as presented in U.S. patent application Ser. No. 15/276,337. In addition to the previous descriptions, note that FIG. 5 presents the file name as medicinal treatment names written with a dash between them. In the case of generic names having more than one word in the name describing the treatment, for example calcium carbonate, the names are not directly combined as though one word but also contain a dash. Note that FIG. 8 presents indications with capital letters for clarification purposes, though capital letters need not be utilized.

With respect to FIG. 9, note that a provider, or even a member of the public reviewing the template, would likely identify that the PRN use of omeprazole at 6:30 (18:30) is probably not appropriate given the PRN use at 7 pm (19:00). In such a case, a provider would likely describe this concern in the special considerations section placed in the “Monitoring/S.C.” field of the template, or create another treatment plan, or direct a recipient to another pre-existing template. Nonetheless, it is possible a provider could prescribe this combination, shown here as an example, either based on empiric evidence or as an experimental approach, which may not yet be described in standard medical literature. However, such a potential abnormality of combinations could be identified as a potential problem electronically

What is claimed is:

1. A method of organizing medicinal treatment combinations, wherein each combination becomes an electronic file, comprising the steps of:
a. arranging in sequence at least two complete names of a medicinal treatment in accordance with their administration time, wherein treatments are arranged from earliest times in a 24 hour period to latest times, wherein a prescription medicine or supplement is described by its full generic name, and wherein a plant or living organism is described by its full Latin name, wherein the first letter of the first word of said name when said name comprises more than one word receives alphabetical priority, wherein a medicinal treatment comprising more than one word has each word joined together through the use of a dash character, and wherein the at least two complete names are separated by a dash;
b. entering and saving the arrangement of names as a file name into a software program for development according to the skill of the art, wherein said electronic file name comprises a URL for directing to a webpage hosted on a server;
c. uploading the aforementioned file to said server, whereby it can be found, viewed and edited by healthcare providers or members of the public in accordance with the skill of the art of software programs for webpage management, wherein information relevant to the appropriateness, safety, effectiveness, risks and benefits of the combination of medicinal treatments is added to the webpage according to fields of information arranged as columns corresponding to medicinal treatments arranged as rows, wherein said columns consist of:
1. the name of the medicine or supplement, its strength, and any prescription number;
2. the date it was prescribed or first started, as well as the prescriber if applicable;
3. the manufacturer, shape, color and any markings on the dosage form
4. the indication of the treatment or why it was prescribed or what it is used for;
5. monitoring parameters and special considerations, including contraindications;
6. the intended result the treatment produces or should produce or treatment goals any contraindication;
7. possible side effects.

2. The method of claim 1 wherein each medicinal treatment is arranged alphabetically instead of by administration time.

2. A method of organizing medicinal treatment combinations, wherein each combination becomes an electronic file, comprising the steps of:
a. arranging all the medicinal treatments of a person or animal into groups based on their administration time during a specific time of day, wherein the hour of day is accorded a numerical value between 1 and 24, wherein the value for the hour is followed by the letter M and said M is followed by a number between 1 and 60 to correspond with one of the 60 minutes in an hour whenever an administration time does not begin on the hour, wherein the value for the minute is followed by the letter S, and said S is followed by a number between 1 and 60 to correspond with one of the 60 seconds in a minute when an administration time does not begin on the minute;
b. arranging alphabetically all the medicinal treatments of a person or animal administered at a time corresponding to one of said groups, wherein each treatment is described by its full generic name in the case of a prescription medicines or over-the-counter substance, or its full Latin name when said substance is derived from a plant or other living organism, and wherein each word of a medicinal treatment is separated by a dash character;
c. placing the numeric value corresponding to an administration time before the corresponding alphabetically-arranged group;
a. combining, entering and saving the arrangement of administration times and names as a file name in a software program for webpage development according to the skill of the art, wherein said electronic file name comprises a URL for directing to a webpage hosted on a server;
a. uploading the aforementioned file to said server, wherein information relevant to the appropriateness, safety and effectiveness, risks and benefits of the specific combination of medicinal treatments is added to the webpage according to fields of information arranged as columns corresponding to medicinal treatments arranged as rows, wherein said columns consist of:
i. the name of the medicine or supplement, its strength, and any prescription number;
ii. the date it was prescribed or first started, as well as the prescriber if applicable;
iii. the manufacturer, shape, color and any markings on the dosage form;
iv. the indication of the treatment or why it was prescribed or what it is used for;
v. monitoring parameters and special considerations, including contraindications;
vi. the intended result the treatment produces or should produce or treatment goals any contraindication;
vii. possible side effects.

3. The method of claim 1 wherein at least one medicinal treatment is preceded by the of indication for the medicinal treatment, wherein a dash character precedes and follows said indication.

4. The method of claim 1 wherein each word is separated by at least one special character other than a dash character.

5. The method of claim 1 wherein each medicinal treatment is abbreviated according to a commonly used medicinal treatment abbreviation.

6. The method of claim 1 wherein each word in the name of a medicinal treatment is abbreviated to the fewest letters of each word in its name that would be necessary to form a distinct abbreviation for that medicinal treatment among all known medicinal treatments.

7. The method of claim 1 wherein each medicinal treatment is arranged alphabetically instead of by administration time.

8. The method of claim 1 wherein at least one medicinal treatment is preceded by the of indication for the medicinal treatment, wherein a dash character precedes and follows said indication.

9. The method of claim 1 wherein each word is separated by at least one special character other than a dash character.

10. The method of claim 1 wherein each medicinal treatment is abbreviated according to a commonly used medicinal treatment abbreviation.

11. The method of claim 1 wherein each word in the name of a medicinal treatment is abbreviated to the fewest letters of each word in its name that would be necessary to form a distinct abbreviation for that medicinal treatment among all known medicinal treatments.
